(19) United States
(12) Patent Application Publication
(10) Pub. No.: US 2009/0192603 A1
(43) Pub. Date: Jul. 30, 2009

(54) Adjustable Sizer Devices for Minimally Invasive Cardiac Surgery

(75) Inventor: Timothy R. Ryan, Shorewood, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/358,841

(22) Filed: Jan. 23, 2009

Related U.S. Application Data
(60) Provisional application No. 61/062,414, filed on Jan. 25, 2008.

(51) Int. Cl. A61F 2/26; A61B 19/00

(52) U.S. Cl. 623/2.11; 128/898

(57) ABSTRACT

Described is an adjustable device for sizing a heart valve annulus by a minimally invasive route, the device comprising: a cannula comprising a proximal end, a distal end and an interior lumen; and at least one wire extending through the interior lumen of the cannula, wherein the at least one wire may be advanced or retracted through the lumen and from the distal end of the cannula, such that a segment of the wire may form a plurality of different predetermined shapes of predetermined sizes used to size the heart valve annulus. Additionally, methods of using such adjustable sizing devices are disclosed.

### PRIORITY

The present non-provisional patent application claims benefit from U.S. Provisional Patent Application having Ser. No. 61/062,414, filed on Jan. 25, 2008, by Kuehn et al., and titled SIZER, HOLDER AND DELIVERY DEVICES FOR MINIMALLY INVASIVE ANNULOPLASTY SURGERY, wherein the entirety of said provisional patent application is incorporated herein by reference.

### TECHNICAL FIELD

This invention generally relates to devices and methods of repair and replacement of heart valves. In particular, the invention relates to devices for measuring the size of a heart valve annulus and for holding and delivering an annuloplasty device to the annulus during minimally invasive cardiac surgery.

### BACKGROUND OF THE INVENTION

Heart valve disease is a widespread condition in which one or more of the valves of the heart fails to function properly. Various surgical techniques may be used to replace or repair a diseased or damaged valve. Damaged leaflets of the valve may be excised and the annulus sculpted to receive a replacement valve. Another less drastic method for treating defective valves is repair or reconstruction by annuloplasty, in which the effective size of the valve annulus is contracted and reinforced, by attaching a prosthetic annuloplasty ring or band to an interior wall of the heart around the valve annulus. The annuloplasty ring or band is designed to support the functional changes that occur during the cardiac cycle, while maintaining leaflet coaptation and valve integrity.

To perform successful valve replacement and annuloplasty surgeries, the size of the valve annulus must be accurately measured. Sizing may be achieved by measuring the width and the height of the anterior leaflet of the mitral valve, for example, using sizing obturators. Another way to size the annulus is to use valve sizers, which resemble the shape of the valve annulus and are provided in various sizes. In order to use valve sizers, a surgeon estimates the valve annulus size and selects a sizer accordingly. The sizer is guided into proximity of the annulus using a handle. If the sizer is not judged to be the correct size, it is withdrawn, and replaced by a different sizer. Once the size of the annulus has been determined, a properly sized valve or annuloplasty device may be selected. The selected annuloplasty device is placed on a holder device that is delivered to the annulus. The annuloplasty device is attached to the annulus and removed from the holder device. The delivery device and holder device are then removed from the body.

Surgical techniques for annuloplasty surgery are typically performed open-chest. This usually requires the patient to be placed on a cardiac bypass machine to pump and oxygenate the blood while the surgeon operates on the stopped heart muscle. Open-chest surgery can be very traumatic on the patient and recovery can take many months. Additionally, such surgery may not be an option for some patients due to limited possibility for recovery, concurrent disease, or age.

For these reasons, it is desirable to use minimally invasive cardiac surgical techniques for valve repair. However, these procedures reduce the available space to deliver surgical instruments to a surgical site, and reduce the space in which surgical instruments may be operated within the area of the surgical site. Therefore, such procedures require surgical instruments with appropriate size and maneuverability that accommodate the limited space.

Traditional annuloplasty and valve sizing and holding instruments were designed for use with open-chest surgery that exposes the appropriate regions of the heart to complete and open access through the open chest wall. The ability of these instruments to fit through significantly reduced surgical field access points was not a necessary criteria for their design. Advances in the surgical field toward minimally invasive techniques has created significant new challenges for the design of new instruments and the development of new techniques for using these instruments to successfully complete procedures in limited access surgical fields.

Exemplary types of minimally invasive cardiac surgery include atrio-ventricular valve repair, reconstruction, or replacement surgical procedures. In particular, the replacement of the valves and repair of valve annulus dilation using annuloplasty devices can employ minimally invasive techniques.

Despite the current existence of sizing devices for sizing a valve annulus, there is still a need for improved devices, and in particular those devices that may be used during minimally invasive cardiac surgical procedures.

### SUMMARY OF THE INVENTION

Embodiments of the present invention include sizer devices that are made, configured and/or may be manipulated to fit through significantly reduced surgical field access points and may be used in reduced surgical fields of operation. In particular, the sizer devices are adjustable and may obtain different configurations corresponding to a plurality of heart valve annulus sizes. Additionally, the devices are adjustable to be able to size annuluses of different sizes. The devices include segments that are retractable in order to allow the devices to fit through reduced surgical field access points.

The embodiments of the present invention offer an advantage that they may be used during minimally invasive cardiac surgery to fit through significantly reduced surgical field access points and in reduced surgical fields of operation. In doing so, the embodiments of the present invention reduce the physical trauma to the patient by eliminating the need to perform a complete sternotomy, and reduce the time spent in surgery. The embodiments of the present invention also allow annuloplasty surgery to be performed on patients that would not otherwise be able to have the surgery involving open-chest techniques. The embodiments of the present invention also reduce the time spent in surgery, in that each device is adjustable and can obtain a plurality of sizes, which eliminates the need for a surgeon to have to insert and remove a plurality of different sizing devices. Furthermore, the embodiments of the present invention allow for maximum visibility of valve structure in the surgical field.

A first aspect of the present invention is an adjustable device for sizing a heart valve annulus by a minimally invasive route. A first embodiment of the device comprises: a cannula comprising a proximal end, a distal end and an interior lumen; and at least one wire extending through the interior lumen of the cannula, wherein the at least one wire may be advanced or retracted through the lumen and from the distal end of the cannula, such that a segment of the wire may form a plurality of different predetermined shapes of predetermined sizes used to size the heart valve annulus. The at least one wire may be completely retracted into the interior lumen in order for the device to be inserted and removed from a body through a minimally invasive route. The plurality of different predetermined shapes or predetermined sizes correspond to annuloplasty devices having the same shapes and sizes. The at least one wire may be controlled from the proximal end of the cannula in order to be extended and retracted. The at least one wire may comprise a shape memory alloy. The segment of the at least one wire that extends from the distal end of the cannula to form the predetermined shapes of predetermined sizes can extend generally perpendicular to the cannula. The plurality of different predetermined shapes of predetermined sizes may correspond to stented tissue cardiac valve devices, stentless tissue cardiac valve devices or mechanical cardiac valve device having the same shapes and sizes.

A second embodiment of the first aspect of the present invention may comprise: a cannula comprising a proximal end, a distal end and an interior lumen; and a plurality of wires comprising proximal and distal ends and extending through the interior lumen of the cannula, wherein the plurality of wires may be advanced or retracted together through the lumen and from the distal end of the cannula, such that the distal ends of the plurality of wires may form one of a plurality of different predetermined shapes and may be spaced apart. The plurality of wires may be completely retracted into the interior lumen in order for the device to be inserted and removed from a body through a minimally invasive route. The plurality of different predetermined shapes or predetermined sizes correspond to annuloplasty devices having the same shapes and sizes. The plurality of wires may be controlled from the proximal end of the cannula in order to be extended and retracted. The plurality of wires may comprise a shape memory alloy. The plurality of different predetermined shapes of predetermined sizes may correspond to stented tissue cardiac valve devices, stentless tissue cardiac valve devices, or mechanical cardiac valve devices having the same shapes and sizes.

A second aspect of the present invention is a method of sizing a heart valve annulus. One embodiment of the method comprises the steps of: receiving an adjustable device for sizing a heart valve annulus by a minimally invasive route, the device comprising: a cannula comprising a proximal end, a distal end and an interior lumen; and at least one wire extending through the interior lumen of the cannula, wherein the at least one wire may be advanced or retracted through the lumen and from the distal end of the cannula, such that a segment of the wire may form a plurality of different predetermined shapes of predetermined sizes used to size the heart valve annulus; inserting the device into the minimally invasive route; advancing the at least one wire from the distal end of the cannula such that the advanced segment forms a first shape and size of the plurality of predetermined shapes and sizes; comparing the advanced segment of wire to the heart valve annulus; if the advanced segment fits the heart valve annulus, then retracting and removing the device from the minimally invasive route; and if the advanced segment does not fit the heart valve annulus, then advancing or retracting the at least one wire such that the advanced segment forms a second shape and size of the plurality of predetermined shapes and sizes, and repeating until the advanced segment fits the heart valve annulus, then retracting and removing the device from the minimally invasive route.

A second embodiment of the second aspect of the present invention comprises the steps of: receiving an adjustable device for sizing a heart valve annulus by a minimally invasive route, the device comprising: a cannula comprising a proximal end, a distal end and an interior lumen; and a plurality of wires comprising proximal and distal ends and extending through the interior lumen of the cannula, wherein the plurality of wires may be advanced or retracted together through the lumen and from the distal end of the cannula, such that the distal ends of the plurality of wires may form one of a plurality of different predetermined shapes and may be spaced apart; inserting the device into the minimally invasive route; advancing the plurality of wires from the distal end of the cannula such that the distal ends of the plurality of wires form a first shape and are spaced apart a first distance; comparing the distal ends of the plurality of wires the heart valve annulus; if the distal ends of the plurality of wires fit the heart valve annulus, then retracting and removing the device from the minimally invasive route; and if the distal ends of the plurality of wires do not fit the heart valve annulus, then advancing or retracting the plurality of wires such that the distal ends of the wires form a second shape and are spaced apart a second distance, and repeating until the distal ends of the wires fit the heart valve annulus, then retracting and removing the device from the minimally invasive route.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of a distal end portion of an adjustable sizing device, in accordance with the present invention;

FIG. 2 is a side view, and partially see-through, of a distal end portion of an adjustable sizing device, in accordance with the present invention; and

FIG. 3 is a distal end view of the device of FIG. 2.

### DETAILED DESCRIPTION OF PREFERRED EMBODIMENTS

Embodiments of the present invention include sizer devices that are made, configured and/or may be manipulated to fit through significantly reduced surgical field access points and may be used in reduced surgical fields of operation. In particular, the sizer devices are adjustable and may obtain different configurations corresponding to a plurality of heart valve annulus sizes and/or shapes. Additionally, an adjustable portion of the devices that is able to size annuluses is retractable in order to allow the devices to fit through reduced surgical field access points. Particularly, the sizer devices will be discussed with regard to their use during annuloplasty surgery. Although the sizer devices shown may be best used to size a mitral valve annulus, for example, it is contemplated that the present invention may be configured to be used to size any of the heart valve annuli. Also, although the present application addresses annuloplasty surgery, it is contemplated that the present invention or features thereof may be used during other minimally invasive surgical procedures as well.

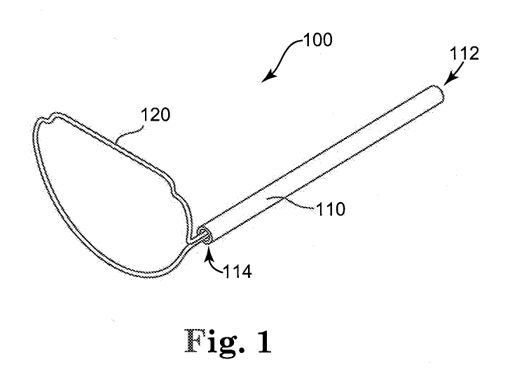

With reference to the accompanying figures, wherein like components are labeled with like numerals throughout the several figures, and, initially, to FIG. 1, a perspective view of a distal portion of a sizer device 100 in accordance with the present invention is illustrated. Sizer device 100 preferably comprises a cannula 110 (with an interior lumen 112) through which a wire 120 may be delivered adjacent an annulus, for example. The wire 120 may be advanced through the cannula 110 and out the distal end 114 in varying amounts by which a plurality of sizes and shapes of wire 120 may be formed. The shape formed by the wire 120 in FIG. 1 may correspond to a device used in a mitral valve annulus, for example. The wire 120 of sizing device 100 preferably is able to obtain shapes that correspond to annuloplasty devices used in different heart valve annuli (e.g., the mitral valve).

It is contemplated that many different sizes and shapes may be formed by the wire 120 besides those illustrated in the figures. A circular shape may, for example, be formed for sizing applications in the pulmonary and aortic valve positions. Alternatively, a kidney shape corresponding to the tricuspid annulus shape may be formed for tricuspid valve annulus sizing. The shape could also include three-dimensional shape for sizing tricuspid and mitral valves, for example, which may correspond to the natural anatomical shape of the atrio-ventricular annuli.

The wire 120 may extend from the distal end 114 of the cannula 110 in order to form a desired predetermined shape and size. FIG. 1 shows the wire 120 extending generally perpendicular to the length of the cannula 110 from the distal end 114. This arrangement of the wire 120 allows the cannula 110 of the device 100 to be inserted generally perpendicularly to the valve annulus in order to measure or size the annulus. However, it is contemplated that the wire 120 may extend at any possible angle from the lengthwise direction of the cannula 110, including parallel to the cannula 110.

The plurality of different predetermined shapes of predetermined sizes preferably correspond to annuloplasty devices having the same shapes and sizes. Alternatively, however, the predetermined shapes and sizes may, for example, correspond to stented tissue cardiac valve devices, stentless tissue cardiac valve devices, or mechanical cardiac valve devices having the same shapes and sizes.

Preferably, the wire 120 is advanced and retracted through the lumen 112 of the cannula 110, and is controlled remotely from a proximal end (not shown) of sizing device 100 by a user. It is contemplated that many different means for advancing and retracting the wire 120 are possible. Another exemplary way of advancing and retracting the wire 120 may be to hold the cannula 110 fixed while advancing or retracting a second inner cannula (not shown) to which the wire 120 is anchored. Yet another exemplary way to advance or retract the sizer wire 120 may be to use a threaded handle (not shown) that is twisted in order to advance or retract the wire 120. A further exemplary way would be to use a syringe piston (not shown) to extend or retract the wire 120. Additionally, a trigger (not shown) could be pulled in order to advance the wire 120, which may include a locking feature (also not shown) that may be released to retract the wire 120. Also, a handle (not shown) may be squeezed in order to advance the wire 120, with relaxing of the handle causing retraction. In all described scenarios for advancing and retracting the wire 120, an option of incorporating a locking feature that retains the advanced or retracted state of the wire 120 is also contemplated.

The wire 120 may comprise a metal or other suitable material. Preferably the wire 120 comprises a material having shape memory, such as Nitinol™. Therefore, when the wire 120 extends out of the cannula 110, a predetermined shape is able to be formed.

The wire 120 may be advanced a plurality of predetermined amounts in order to form shapes that correspond to more than one size and shape of heart valve annulus, for example. Preferably, the wire 120 may be able to obtain the size and shape of a family of annuloplasty devices. An advantage of sizer device 100 is that only one device needs to be inserted into a surgical port in order to size a valve annulus, rather than multiple devices.

Sizing device 100 may be used in a minimally invasive annuloplasty surgery to size a mitral valve annulus, for example. First, the device 100 is inserted through a reduced surgical access site and delivered to a desired position adjacent the valve annulus. Preferably, the device 100 is delivered with the wire 120 in a retracted position, such that the wire 120 does not extend out the distal end 114 of the cannula 110 and is located inside the interior lumen 112 of the cannula 110. Next, the wire 120 is advanced out the distal end 114 of the cannula 110 a predetermined amount in order to form a shape of a given size that corresponds to an annuloplasty device. The shape of the wire 120 is then compared to the annulus to determine whether or not the size and shape are correct. If so, the wire 120 is preferably retracted, and the sizing device 100 is removed. If the size and shape are not correct, however, the wire 120 may be advanced or retracted to form other shapes and/or other sizes that are compared to the annulus until an appropriate shape and/or size are chosen, at which time the wire 120 is retracted into the lumen 112 and the sizing device 100 is removed.

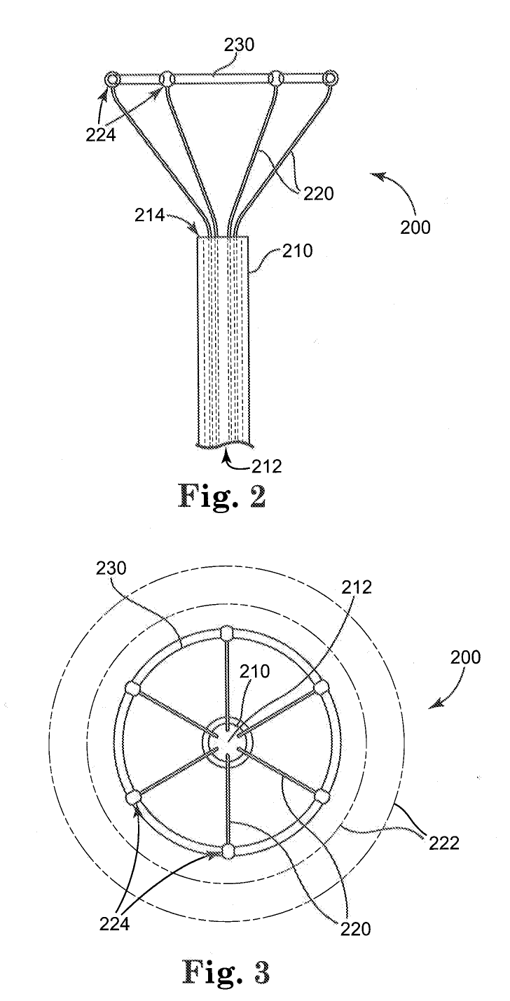

FIGS. 2 and 3 illustrate a side view and distal end view, respectively, of another embodiment of a sizer device 200, in accordance with the present invention. The sizer device 200 comprises a cannula 210 having an interior lumen 212 and a plurality of wires 220. The plurality of wires 220 are advanced out the distal end 214 of the cannula 210 and spread out to preferably form a predetermined shape also preferably having a predetermined size, in order to size a mitral valve annulus, for example.

Preferably, the wires 220 comprise a material having shape memory, such as Nitinol™. The plurality of wires 220 form a predetermined shape, as shown in FIG. 3, from a distal end view. A circular shape is shown, but other shapes are also contemplated.

The distal ends 224 of the wires 220 are preferably covered with some material in order to avoid puncture of bodily tissue by the wires 220 when extended from cannula 210. Some exemplary materials used to cover the distal ends 224 of the wires 220 include, but are not limited to, elastomeric materials, such as epoxies, urethanes and silicones. Other materials that are also contemplated include fabrics, such as polyester fabric. In addition, it is possible to compound the silicone and polymers, for example, with metallic materials to give the distal ends 224 radiopaque characteristics.

It is contemplated that the wires 220 may be extended different amounts from the cannula 210, which may correspond to different sizes and shapes of valve annuli. The figure shows, by dashed lines 222, two other possible configurations of the wires 220, resulting in different diameters or sizes of the sizing segment of the device.

FIGS. 2 and 3 show the wires 220 extended and surrounded by an optional elastic band 230. The purpose of the elastic band 230 is to keep the ends 224 of the plurality of wires 220 in the desired configuration. The elastic band 230 is preferably made of an elastomeric material, but other materials are also contemplated. Other means for surrounding or outlining the perimeter of the wires 220 when extended are also contemplated.

Preferably, the wires 220 are advanced and retracted through the lumen 212 of the cannula 210 and controlled remotely from a proximal end (not shown) of sizing device 200 by a user. It is contemplated that many different means for advancing and retracting the wires 220 are possible. Another exemplary way of advancing and retracting the wires 220 may be to hold the cannula 210 fixed while advancing or retracting a second inner cannula (not shown) to which the wires 220 are anchored. Yet another exemplary way to advance or retract the wires 220 may be to use a threaded handle (not shown) that is twisted in order to advance or retract the wires 220. A further exemplary way would be to use a syringe piston (not shown) to extend or retract the wires 220. Additionally, a trigger (not shown) could be pulled in order to advance the wires 220, which may include a locking feature (also not shown) that may be released to retract the wires 220. Also, a handle (not shown) may be squeezed in order to advance the wires 220, with relaxing of the handle causing retraction. In all described scenarios for advancing and retracting the wires 220, an option of incorporating a locking feature that retains the advanced or retracted state of the wires 220 is also contemplated.

The wires 220 may be advanced predetermined amounts in order to form shapes that correspond to more than one size and shape of heart valve annulus, for example. Preferably, the wires 220 may be able to obtain the size and shape of a family of annuloplasty devices. An advantage of sizer device 200 is that only one device needs to be inserted into a surgical port in order to size a valve annulus, rather than multiple devices.

Sizing device 200 may be used in a minimally invasive annuloplasty surgery to size a mitral valve annulus, for example. First, the device 200 is inserted through a reduced surgical access site and delivered to a desired position adjacent the valve annulus. Preferably, the device 200 is delivered with the wires 220 in a retracted position, such that the wires 220 do not extend out the distal end 214 of the cannula 210 and are located inside the interior lumen 212 of the cannula 210. Next, the wires 220 (possibly with optional elastic band 230 attached) are advanced out the distal end 214 of the cannula 210 a predetermined amount in order to form a shape of a given size that corresponds to an annuloplasty device. The wires 220 are then compared to the annulus to determine whether or not the size and shape are correct. If so, the wires 220 are preferably retracted, and the sizing device 200 is removed. If the size and shape are not correct, however, the wires 220 may be advanced or retracted to form other shapes or other sizes that are compared to the annulus until an appropriate shape and/or size are chosen, at which time the wires 220 are retracted into the lumen 212 and the sizing device 200 is removed.

It is to be understood that while particular embodiments of the invention have been illustrated for use in typical valve repair procedures, various modifications to shape, and arrangement of parts can be made as may be desirable for varying applications as may relate to valve sizes or later developed techniques. The invention should not be considered limited to the specific methods and devices precisely described herein. On the contrary, various modifications will be apparent to those of ordinary skill upon reading the disclosure. Although certain embodiments are described with reference to the mitral valve, use with other valves or anatomical structures is also contemplated. The foregoing detailed description has been given for clarity of understanding only. No unnecessary limitations are to be understood there from. The entire disclosure of any article, patent or patent application identified herein is hereby incorporated by reference.

1. An adjustable device for sizing a heart valve annulus by a minimally invasive route, the device comprising:
a cannula comprising a proximal end, a distal end and an interior lumen; and
at least one wire extending through the interior lumen of the cannula, wherein the at least one wire may be advanced or retracted through the lumen and from the distal end of the cannula, such that a segment of the wire may form a plurality of different predetermined shapes of predetermined sizes used to size the heart valve annulus.

2. The adjustable sizing device of claim 1, wherein the at least one wire may be completely retracted into the interior lumen in order for the device to be inserted and removed from a body through a minimally invasive route.

3. The adjustable sizing device of claim 1, wherein the plurality of different predetermined shapes or predetermined sizes correspond to annuloplasty devices having the same shapes and sizes.

4. The adjustable sizing device of claim 1, wherein the at least one wire is controlled from the proximal end of the cannula in order to be extended and retracted.

5. The adjustable sizing device of claim 1, wherein the at least one wire comprises a shape memory alloy.

6. The adjustable sizing device of claim 1, wherein the segment of the at least one wire that extends from the distal end of the cannula to form the predetermined shapes of predetermined sizes can extend generally perpendicular to the cannula.

7. The adjustable sizing device of claim 1, wherein the plurality of different predetermined shapes of predetermined sizes correspond to stented tissue cardiac valve devices having the same shapes and sizes.

8. The adjustable sizing device of claim 1, wherein the plurality of different predetermined shapes of predetermined sizes correspond to stentless tissue cardiac valve devices having the same shapes and sizes.

9. The adjustable sizing device of claim 1, wherein the plurality of different predetermined shapes of predetermined sizes correspond to mechanical cardiac valve devices having the same shapes and sizes.

10. An adjustable device for sizing a heart valve annulus by a minimally invasive route, the device comprising:
a cannula comprising a proximal end, a distal end and an interior lumen; and
a plurality of wires comprising proximal and distal ends and extending through the interior lumen of the cannula, wherein the plurality of wires may be advanced or retracted together through the lumen and from the distal end of the cannula, such that the distal ends of the plurality of wires may form a plurality of different predetermined shapes and may be spaced apart.

11. The adjustable sizing device of claim 10, wherein the plurality of wires may be completely retracted into the interior lumen in order for the device to be inserted and removed from a body through a minimally invasive route.

12. The adjustable sizing device of claim 10, wherein the plurality of different predetermined shapes or predetermined sizes correspond to annuloplasty devices having the same shapes and sizes.

13. The adjustable sizing device of claim 10, wherein the plurality of wires are controlled from the proximal end of the cannula in order to be extended and retracted.

14. The adjustable sizing device of claim 10, wherein the plurality of wires comprise a shape memory alloy.

15. The adjustable sizing device of claim 10, wherein the plurality of different predetermined shapes of predetermined sizes correspond to stented tissue cardiac valve devices having the same shapes and sizes.

16. The adjustable sizing device of claim 10, wherein the plurality of different predetermined shapes of predetermined sizes correspond to stentless tissue cardiac valve devices having the same shapes and sizes.

17. The adjustable sizing device of claim 10, wherein the plurality of different predetermined shapes of predetermined sizes correspond to mechanical cardiac valve devices having the same shapes and sizes.

18. A method of sizing a heart valve annulus, the method comprising the steps of:
receiving an adjustable device for sizing a heart valve annulus by a minimally invasive route, the device comprising:
a cannula comprising a proximal end, a distal end and an interior lumen; and
at least one wire extending through the interior lumen of the cannula, wherein the at least one wire may be advanced or retracted through the lumen and from the distal end of the cannula, such that a segment of the wire may form a plurality of different predetermined shapes of predetermined sizes used to size the heart valve annulus;
inserting the device into the minimally invasive route;
advancing the at least one wire from the distal end of the cannula such that the advanced segment forms a first shape and size of the plurality of predetermined shapes and sizes;
comparing the advanced segment of wire to the heart valve annulus;
if the advanced segment fits the heart valve annulus, then retracting and removing the device from the minimally invasive route; and
if the advanced segment does not fit the heart valve annulus, then advancing or retracting the at least one wire such that the advanced segment forms a second shape and size of the plurality of predetermined shapes and sizes, and repeating until the advanced segment fits the heart valve annulus, then retracting and removing the device from the minimally invasive route.

19. A method of sizing a heart valve annulus, the method comprising the steps of:
receiving an adjustable device for sizing a heart valve annulus by a minimally invasive route, the device comprising:
a cannula comprising a proximal end, a distal end and an interior lumen; and
a plurality of wires comprising proximal and distal ends and extending through the interior lumen of the cannula, wherein the plurality of wires may be advanced or retracted together through the lumen and from the distal end of the cannula, such that the distal ends of the plurality of wires may form a plurality of different predetermined shapes and may be spaced apart;
inserting the device into the minimally invasive route;
advancing the plurality of wires from the distal end of the cannula such that the distal ends of the plurality of wires form a first shape and are spaced apart a first distance;
comparing the distal ends of the plurality of wires the heart valve annulus;
if the distal ends of the plurality of wires fit the heart valve annulus, then retracting and removing the device from the minimally invasive route; and
if the distal ends of the plurality of wires do not fit the heart valve annulus, then advancing or retracting the plurality of wires such that the distal ends of the wires form a second shape and are spaced apart a second distance, and repeating until the distal ends of the wires fit the heart valve annulus, then retracting and removing the device from the minimally invasive route.
